(19) United States
(12) Patent Application Publication
(10) Pub. No.: US 2020/0085898 A1
(43) Pub. Date: Mar. 19, 2020

(54) CUCUMIS SATIVUS L. EXTRACT FOR USE IN THE TREATMENT AND/OR PREVENTION OF ENTEROPATHIES, PARTICULARLY OF INFLAMMATORY ORIGIN, IN PETS, AND FEED CONTAINING SUCH EXTRACT

(71) Applicant: NATURALEA SA, Vezia (CH)

(72) Inventor: Bruno Boggiani, Vezia (CH)

(21) Appl. No.: 16/494,187

(22) PCT Filed: Mar. 14, 2018
(86) PCT No.: PCT/IB2018/051690

§ 371 Date: Sep. 13, 2019

(30) Foreign Application Priority Data
Mar. 14, 2017 (IT) 102017000028323

(51) Int. Cl. A61K 36/42; A23K 10/30; A23K 50/40

(52) U.S. Cl. CPC A61K 36/42; A23K 50/40; A23K 10/30

(57) ABSTRACT

A Cucumis Sativus L. extract is disclosed herein, which is designed for use in the treatment and/or prevention of enteropathies, particularly of inflammatory origin, in pets, and more particularly enteropathies in dogs.

### DESCRIPTION

The present invention relates to a plant extract for use in the treatment and/or prevention of enteropathies in pets, particularly enteropathies in dogs.

Particularly, the present invention relates to a feed containing such plant extract for treatment and/or prevention of enteropathies in pets, particularly enteropathies in dogs.

Enteropathy is known to be an inflammatory disease of the intestine.

Namely, when such disorder occurs, the stomach lining is infiltrated with inflammatory cells, and normal absorptive and digestive functions are thus impaired.

In dogs, the symptoms of this disease generally include irregular bouts of diarrhea, loss of appetite, vomiting or altered stool, i.e. Stool with mucus.

The term IBD (Inflammatory Bowel Disease) enteropathy is intended to designate a group of chronic idiopathic enteropathies which are classified according to their location and inflammatory histopathology, as well as their partial or no response to dietary, antiparasitic and/or antibiotic therapies. The clinical picture of IBD depend on the affected gastroenteric tract, the clinical phase (remission and/or relapse of the disease), as well as any complication associated with a concurrent protein-dependent enteropathy and/or the lack of certain nutrients (folates and cobalamin).

IBD is one of the most common causes of chronic diarrhea and vomiting in adult dogs.

IBD cases have been also found in dogs fed with packaged feed of unknown origin, processing and storage, particularly feed that has been treated with or contains chemical additives.

This diseases leads with time to an alteration of the morphology of intestinal villi in the dog, at the inflamed ileum.

No particular therapy currently exists for treatment or prevention of enteropathy in dogs.

Accordingly, enteropathy in dogs is currently handled by:

prescribing a special diet for the animal,

protecting the animal from physical stress and, in the most serious cases

administering prednisone, corticosteroids and/or antibiotics to the animal.

Nevertheless, it will be apparent that these measures for treating enteropathies lead to a number of drawbacks such as, for example, water retention problems and alterations of the intestinal flora in dogs, as well as the possibility of administering the aforementioned treatments for a limited period of time, particularly concerning the administration of the aforementioned substances.

In view of the foregoing, it will be understood that a strong need is currently felt in the market for a medicament for treatment or prevention of enteropathies in dogs that can be used for a long time and is capable of restoring the proper morphology of the intestinal wall of the animals that suffer from enteropathies.

Therefore, the invention is based on the problem of providing a medicament that can meet the above needs, while obviating the prior art drawbacks.

In a further aspect, the invention is based on the problem of being able to treat or prevent enteropathies in dogs, as well as providing a dog feed that can meet the above needs, while obviating the prior art drawbacks.

According to the invention, the aforementioned problems have been solved by a Cucumis Sativus L. extract for use as defined in claim 1, a pharmaceutical composition or a dietary supplement as defined in claim 9 or pet feed as claimed in claim 14.

The applicant surprisingly found that the aforementioned prior art drawbacks can be obviated or prevented when a Cucumis Sativus L. extract is used for the treatment and/or prevention of enteropathy in pets, particularly enteropathy in dogs.

Therefore, the present invention first relates to a Cucumis Sativus L. extract for use in the treatment and/or prevention of enteropathy in pets, particularly enteropathy in dogs.

The invention further relates to a medicament for treating enteropathy in dogs.

In a preferred embodiment of the present invention, the aforementioned medicament only consists of Cucumis Sativus L. extract in powder form.

In a second embodiment of the present invention, the aforementioned medicament consists of a pharmaceutical composition or a dietary supplement comprising a Cucumis Sativus L. extract, particularly in powder form, in association with at least one pharmaceutically and/or veterinary acceptable carrier and/or excipient, suitable for pets, preferably dogs.

Examples of pharmaceutically and/or veterinary acceptable carriers and/or excipients, suitable for pets, preferably dogs, that can be used according to the present invention are sweetening agents, flavoring agents, coloring agents and/or preservatives.

Preferably, the Cucumis Sativus L. extract of the present invention is administered orally.

For oral administration, the Cucumis Sativus L. extract in powder form or the pharmaceutical composition or dietary supplement food that contains it according to the present invention is preferably formulated in solid preparations such as capsules, tablets, pills, powders or granules.

Advantageously, the solid preparation for use according to the present invention comprises 50 to 800 mg, preferably 100 to 600 mg Cucumis Sativus L. extract in powder form.

The amount of Cucumis Sativus L. extract to be administered may change according to a variety of factors such as the severity of the enteropathy, or the age, weight and sex of the dog.

Preferably, the administered amount of Cucumis Sativus L. extract ranges from about 100 mg/day to 250 mg/die.

Preferably, the duration of the treatment may range from 30 days to 90 days, more preferably from 45 days to 70 days, e.g. about 60 days.

Preferably, the Cucumis Sativus L. extract that is used to prepare the medicament of the present invention is used for the treatment and/or prevention of IBD enteropathy in dogs.

Preferably, as used herein, the term “Cucumis Sativus L. extract” is intended to designate a product obtained by subjecting Cucumis Sativus L. to solvent extraction, preferably with a hydroalcoholic solvent, and subsequent drying, preferably vacuum drying. Preferably, the extraction solvent is composed of water/ethanol, with the ethanol amount ranging from 20% to 80% (v/v). Furthermore, extraction is preferably carried out substantially at room temperature, i.e. a temperature ranging from 15 to 30° C. for a time preferably ranging from 5 to 15 hours.

Preferably, drying is freeze-drying, and is possibly preceded by centrifugation and concentration of the extract, e.g. by membrane filtration.

Cucumis Sativus L. is an edible fruit that belongs to the Cucuritaceae family. It has:

an elongate shape, similar to that of zucchini,

a thick, slightly warty and green skin and

a white juicy and slightly acidic pulp, with seeds in the central part.

Generally, Cucumis Sativus L. contains water (96.4%), carbohydrates (2.8%), proteins (0.4%), fats (0.1%), minerals (0.3%), calcium (0.01%), phosphorus (0.03%), iron (1.5 mg/100 g) and vitamin B (30 IU/100 g). In addition, it also contains enzymes, ascorbic acid, lactic acid, essential oils and other important compounds such as tetracyclic triterpenoids.

Topical use of Cucumis Sativus L. is currently known t have the function of softening the skin and imparting greater elasticity thereto.

Furthermore, in human medicine, topical use of Cucumis Sativus L. is particularly suitable for treating skin swelling under the eyes and sunburns.

The Applicant surprisingly found that a Cucumis Sativus L. extract is effective both in treatment of dogs affected by enteropathy and in prevention of enteropathy in healthy dogs that, for example, are subjected to strong physical stress, and could highlight in this respect that fatigue from intense physical stress may lead to the occurrence of enteropathy problems in dogs.

Therefore, in a preferred embodiment the present invention, the Cucumis Sativus L. extract is used to prepare a medicament for the treatment and therapy of enteropathy in dogs and/or to prevent enteropathy in dogs.

It shall be further noted that most dogs are currently fed with dry or moist packaged feed, as such feed can conveniently provide animals with the substances it needs in the proper amounts and proportions, and is quick to use and easily transported.

Therefore, a second aspect of the present invention is to provide dog feed comprising a Cucumis Sativus L extract, alone or as a pharmaceutical composition or dietary supplement that contains it, as discussed above.

Preferably, the dog feed of the present invention is of dry type. More preferably, it is specially designed for use in dogs having gastrointestinal disorders.

In view of the foregoing, the advantages resulting from the use of the aforementioned Cucumis Sativus L. extract and feed of the invention are self-evident.

A first advantage is that the Cucumis Sativus L. extract is of natural origin and has no side effects for the dog, therefore it can be administered to the animal for a long period of time (e.g. up to 60 days) with no harmful effects.

A second advantage is that the use of the Cucumis Sativus L. extract of the present invention can restore the intestinal wall of the dog, if such wall is altered as a result of IBD.

An additional advantage is that the dog feed containing the Cucumis Sativus L. extract of the present invention can treat or prevent enteropathy in dogs, without requiring any further treatment or different diet.

The present invention will be further illustrated by the following examples, which shall be intended without limitation.

### Experimental Procedure

### EXAMPLE 1

### Preparation of a Cucumis Sativus L. Extract of the Invention

A Cucumis Sativus L. extract for use according to the present invention was prepared from fresh fruits.

These fruits were previously minced, preferably by mechanical means, before being subjected to solvent extraction with a hydroalcoholic water/ethanol solvent, with an ethanol amount ranging from 20% to 80% (v/v), for a time ranging from 5 to 15 hours at a temperature ranging from 15 to 30° C.

Then, the product so obtained was concentrated by membrane filtration and powdered by freeze-drying, at a residual pressure ranging from 0.1 to 1 mBar and at a temperature ranging from +20 to −50° C.

The extract so obtained is a light yellow fine hygroscopic powder, having a slightly sweet taste.

### EXAMPLE 2

### Effectiveness Test on the Cucumis Sativus L. Extract for Use According to the Invention in the Treatment of ISB in Dogs

The effectiveness of the Cucumis Sativus L. extract, e.g. obtained according to Example 1, on IBD in dogs, was assessed in 15 dogs diagnosed with IBD:

The diagnostic criteria for IBD included: persistent gastrointestinal symptoms (3 weeks), no response to diet or therapies with antibiotics, anticholinergics, or gastrointestinal protective agents.

All the tested dogs underwent a complete blood count, biochemical analysis of serum, urine analysis, comprehensive stool analysis and abdominal radiographical examination. Additional tests were also carried out in certain cases, such as radiography, abdominal ultrasound, and serum immunoreactivity concentration measurements. Dogs with hypoproteinemia or with suspected intestinal lymphangiectasia were excluded from the study.

Three groups of 15 dogs each were tested:

a first group was composed of healthy dogs,

a second group was composed of IBD-affected dogs,

a third group was composed of IBD-affected dogs treated with the Cucumis Sativus L. extract of Example 1.

The tested dogs were:

Group of 15 healthy dogs: selected from Golden Retriever, Hisky, Boxer, Jack Russel Terrier, WHW Terrier, German shepherd, Yorkshire Terrier, 4 males and 11 females, aged 5 years and a half on average and having an average weight of 21.4 Kg.

Group of 15 IBD-affected dogs: selected from Epagneul Breton, Chow Chow, Rotweiler, Border Collie, German shepherd, Bolognese, Miniature Schnauzer, Yorkshire Terrier, 6 males and 9 females, aged 6 years on average and having an average weight of 26.6 Kg.

Group of 15 IBD-affected dogs treated with Cucumis Sativus L. of the present invention: selected from Epagneul Breton, Chow Chow, Rotweiler, Border Collie, German shepherd, Bolognese, Miniature Schnauzer, Yorkshire Terrier, 6 males and 9 females, aged 6 years on average and having an average weight of 26.6 Kg.

The dogs of the first group (control group) were judged healthy based on the normal results of a physical examination, on blood cell counts, on a biochemical analysis of serum, on urine analysis, on repeated stool examinations and on the dirofilaria antigen test.

The dogs of the second group were judged to be affected by IBD based on their medical history and on examinations and received no treatment in this test.

The treatment of dogs of the third group consisted in administering 1 tablet/day of the Cucumis Sativus L. extract (150 mg of the Cucumis Sativus L. extract) for 60 consecutive days.

The three groups underwent a 90-day open label test to assess the effects of the Cucumis Sativus L. extract of the present invention on the plasma and on histological markers.

The clinical disease index CIBDAI was assessed at the start time 0 (T0) and 90 days (T1) from the start, i.e. 30 days from the end of the treatment with the Cucumis Sativus L. extract of the present invention.

The CIBDAI index is based on the following six criteria, each having a score from 0 to 3: behavior/activity, appetite, vomiting, consistency of stool, frequency of stool and weight loss.

The sum of the scores is classified as:

clinically insignificant (score from 0 to 3),

mild (score from 4 to 5),

moderate (score from 6 to 8), or

severe (score equal to or higher than 9).

At the start time (time T0) and after 90 days (T1), multiple tissue samples (10-15 samples) of the intestinal mucosa were extracted by endoscopic biopsy from the small intestine and/or the large intestine of the three groups of dogs and were assessed. The tissue samples were obtained directly at the areas of the mucosal lesions in areas with increased granularity, friability or erosion, as well as in apparently normal areas of the mucosa.

A “severe” score was assigned based on the extent of the injury of the epithelial structure and the changes of the mucosa, as recently suggested by WSAVA for gastrointestinal inflammation diagnosis.

The samples (blood and tissue) were collected and stored in glass vials at −80° C. until use.

The samples underwent lipid extraction with chloroform/ethanol with deuterated internal standards.

The organic phase was dried, pre-purified by silica chromatography and analyzed by LC-MS/MS (Liquid Chromatography and Mass Spectrometry).

Quantitative analysis was carried out by recording the selected ion with respect to protonated molecular ions.

The purpose of this study was to carry out a randomized open-label test to compare the microbiological, histological and immunomodulating effects of the Cucumis Sativus L. extract in IBD-affected dogs.

Then, the endocannabinoid system was analyzed in plasma levels both in the healthy dogs, and in those affected by IBD and those affected by IBD and treated with the Cucumis Sativus L. of the present invention.

The analysis of the endocannabinoid system in the plasma of the group of 15 healthy dogs showed the following average values: AEA 1.8±0.1 pmol/ml; 2AG 5.0±0.4 pmol/ml; PEA 22.2±1.4 pmol/ml; and OEA 59.6±5.7 pmol/ml.

The analysis of the endocannabinoid system in the plasma of the group of 15 IBD-affected dogs showed the following average values: AEA 1.6±0.2 pmol/ml; 2AG 16.4±3.6 pmol/ml; PEA 40.9±3.8 pmol/ml; and OEA 52.4±7.7 pmol/ml.

The analysis of the endocannabinoid system in the plasma of the group of 15 IBD-affected dogs treated with the Cucumis Sativus L. of the present invention showed the following average values: AEA 1.9±0.1 pmol/ml; 2AG 8.0±2.0 pmol/ml; PEA 21.8±1.4 pmol/ml; and OEA 58.5±7.9 pmol/ml.

Referring to the endocannabinoid system, EEA is N arachidonoyl ethanolamine, 2-AG is 2-arachidonoyl glycerol, PEA is N-palmitoyl ethanolamine and OEA is cis-9-octadecamide.

The above results show that the plasma-related parameters obtained in the group treated with the Cucumis Sativus L. extract of the present invention are the same as those of the group of healthy dogs.

Conversely, the plasma-related parameters obtained in the group of dogs that received no treatment (second group) are altered, particularly referring to PEA levels, which are much higher than PEA levels of both healthy dogs (first group) and dogs treated with the Cucumis Sativus L. extract of the invention (third group).

To confirm the plasma results in the data of the endocannabinoid system expression, the Applicant analyzed first the endogenous expression of PEA in the intestinal mucosa, as it relates to the gastrointestinal district and has peculiar features that show its important role in the protection and biological rebalancing under chronic phlogistic situations.

In the group of healthy dogs, at the intestinal mucosa level, an average value of 0.74 pmol/mg/D.S.±0.07) was found, which showed a physiological presence of PEA in the gastroenteric tissue of dogs. In IBD-affected dogs, the mucosal PEA levels were found to be increased to about five times (3.58±0.12 pmol/mg) as compared to healthy dogs. This production of PEA at the intestinal mucosa level in response to enteric phlogistic insults is aimed at activating protective factors to counteract the inflammatory condition.

The same type of assessment was carried out on IBD-affected dogs treated with the Cucumis Sativus L. of the present invention to assess whether, as ascertained in plasma levels, such treatment entails a decrease of mucosal PEA levels, which indirectly indicates an improvement of the enteric inflammatory condition.

The average PEA value at the intestinal mucosa level in IBD-affected dogs treated with the Cucumis Sativus L. extract of the present invention was 1.23 pmol/mg (D.S.±0.09), and is a statistically significant result (P<0,05).

The above results show that the treatment with the Cucumis Sativus L. extract of the present invention in IBD-affected animals led to a reduction of the endogenous “on request” production of PEA, which restored expression values to the basal levels typical of healthy animals. Thus, such improvement of the inflammatory condition at the mucosal level does not require the activation of endogenous defense systems, such as the endocannabinoid system.

These results were also clinically confirmed by a CIBDAI assessment of the dog conditions. The treatment with Cucumis Sativus L. was thus able to improve the CIBDAI index of IBD severity, which is based on a general activity score, on the occurrence of vomiting, on weight and appetite loss, on the frequency of stools and stool consistency. At the end of the treatment with Cucumis Sativus L. extract, the CIBDAI index was found to decrease from “moderate to severe” IBD to a “clinically irrelevant” or “mild” disease.

At the start time T0, 57% of the dogs was suffering from severe IBD and 43% from moderate IBD; after 90 days T90 (30 days after the period of administration of the Cucumis Sativus L. extract of the present invention) 71% of the dogs had a “clinically irrelevant” CIBDAI index and 29% had a “mild” IBD level.

For each of the three groups of dogs, the Applicant analyzed the histological appearance of the intestinal mucosa of a healthy dog (first group), an IBD-affected dog (second group) and an IBD-affected dog treated with Cucumis Sativus L. of the present invention (third group).

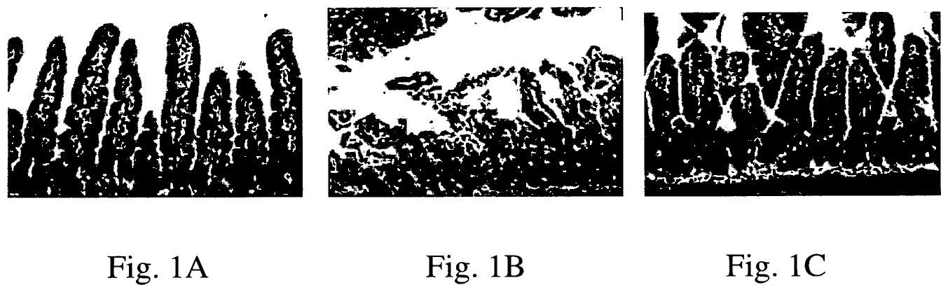

The results so obtained are shown in FIG. 1, in which:

FIG. 1A shows the morphology of the ileum of a healthy dog (first group),

FIG. 1B shows the morphology of the ileum of an IBD-affected dog (second group) and

FIG. 1C shows the morphology of the ileum of an IBD-affected doc at the end of the 60-day period of treatment with the Cucumis Sativus L. extract of the present invention (third group).

As shown in FIG. 1C, the morphology of the ileum of the IBD-affected dog treated with Cucumis Sativus L. of the present invention (FIG. 1C) shows a significant reduction of the histological and structural damage and the inflamed ileum and a good recovery of the proper morphology of intestinal villi.

### EXAMPLE 3

### Effectiveness Test on the Cucumis Sativus L. Extract for Use According to the Invention in the Prevention of ISB in Dogs

The effectiveness of the Cucumis Sativus L. extract, e.g. obtained according to Example 1, on the prevention of IBD in dogs, was assessed by stool analysis in physically stressed dogs.

Intense and extended physical activity often causes gastrointestinal inflammation.

Particularly, in this test nine dogs (selected from the group of Maremmano, Dachsbrake, Bracco German, Setter and Italian hound, aged from 2 to 16 years, with a weight from 13 to 25 Kg, 7 females and 2 males), were subjected to training consisting of wild boar hunting once a day for 30 consecutive days. At the end of this training, their stool was analyzed (T0).

The dogs were later subjected to further agonistic activity, consisting in partridge hunting, twice a week and in 20 minutes' competitions once a week.

Before starting this agonistic activity, the dogs were administered the Cucumis Sativus L. extract of the present invention (1 tablet/day, 150 mg of the Cucumis Sativus L. extract, administered during the only meal of the day, in the evening) for 30 consecutive days. At the end of these 30 days, their stool was analyzed (T1).

The dogs continued their agonistic activity for 90 more days, and their stool was analyzed after the first 30 days (T2) and after 60 days (T3).

The following results were obtained: upon administration of the Cucumis Sativus L. extract for use according to the present invention, the athletic performance of the dogs was found to improve, whereas it was found to decrease when the treatment with the extract was discontinued, the stool of the dogs treated with the Cucumis Sativus L. extract of the present invention (T1) was found to have a better consistency than that before treatment (T0) and after treatment discontinuation (T2, T3).

The Cucumis Sativus L. extract for use according to the present invention was also found to be able to prevent the occurrence of IBS by reducing the cytotoxicity induced by lipopolysaccharide (LPS), the expression of the Toll receptor (TLR-4), of the tumor necrosis factor (TNFα) and the concentration of interleukin IL-8, and to increase the tight junction protein (ZO-1) and the protective heme-oxygenase gene (HO-1).

Those skilled in the art will obviously appreciate that, a number of changes and variants may be made to the experimental procedures as described hereinbefore to meet incidental and specific needs, without departure from the scope of the invention, as defined in the following claims.

1-16. (canceled)

17. A method for the treatment and/or prevention of enteropathies in an animal in need thereof, the method comprising:
administering an effective amount of Cucumis Sativus L. extract to an animal; and
treating and/or preventing enteropathy in the animal.

18. The method of claim 17, wherein said Cucumis Sativus L. extract is in powder form or in the form of a solid preparation.

19. The method of claim 17, wherein said Cucumis Sativus L. extract is administered orally.

20. The method of claim 17, wherein the effective amount of Cucumis Sativus L. extract administered is from 100 mg/day to 250 mg/day.

21. The method of claim 17, wherein said enteropathy is IBD (Inflammatory Bowel Disease).

22. The method of claim 17, wherein the Cucumis Sativus L. extract is obtained by hydroalcoholic solvent extraction of Cucumis Sativus L. fruit.

23. The method of claim 22, further comprising drying the hydroalcoholic solvent extraction so obtained.

24. A pharmaceutical composition or dietary supplement for use in the treatment and/or prevention of enteropathies in animals comprising:
a Cucumis Sativus L. extract; and
at least one pharmaceutically or veterinary acceptable carrier and/or excipient.

25. The pharmaceutical composition or dietary supplement of claim 24, wherein the Cucumis Sativus L. extract is in powder form or in the form of a solid preparation suitable for oral administration.

26. The pharmaceutical composition or dietary supplement of claim 24, further comprising an animal feed source.
